(19) United States
(12) Patent Application Publication
(10) Pub. No.: US 2018/0118892 A1
(43) Pub. Date: May 3, 2018

(54) ACTIVE MATERIALS ENCAPSULATED IN A SOL-GEL DERIVED COMPOSITION AND METHOD OF USE

(71) Applicant: ABS MATERIALS, INC., Wooster, OH (US)

(72) Inventors: Paul L. EDMISTON, Wooster, OH (US); Stacey L. DEAN, Broadview Heights, OH (US)

(21) Appl. No.: 15/572,148

(22) PCT Filed: May 9, 2016
(86) PCT No.: PCT/US16/31534

§ 371 Date: Nov. 6, 2017

Related U.S. Application Data
(60) Provisional application No. 62/159,165, filed on May 8, 2015.

(51) Int. Cl. C08G 77/52; C08G 77/18; B01J 13/00; A61K 8/02; A61K 8/11; A61Q 13/00; A61Q 19/00

(52) U.S. Cl. CPC C08G 77/52; C08G 77/18; B01J 13/00; A61K 8/0279; A61K 2800/624; A61Q 13/00; A61Q 19/00; C08G 2220/00; A61K 2800/56; A61K 8/11

(57) ABSTRACT

Disclosed is encapsulated active material product comprising: an active material encapsulated in a sol-gel derived material, the sol-gel derived material including a plurality of alkylsiloxy substituents and the sol-gel derived material obtained from:

(a) at least one first alkoxysilane precursor having the formula:

(R′O)₃—Si—(CH₂)ₙ—Ar—(CH₂)ₘ—Si—(OR′)₃ (1)

where n and m are individually an integer from 1 to 8, Ar is a single-, fused-, or poly-aromatic ring, and each R′ is independently a C₁ to C₅ alkyl group and

(b) optionally, at least one second precursor having the formula:

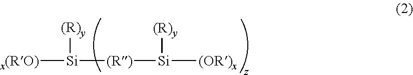

where x is 1, 2, 3 or 4; y is 0, 1, 2, 3; z is 0, 1; the total of x+y+z is 4; each R is independently an organic functional group; each an R′ is independently a C₁ to C₅ alkyl group and R″ is an organic bridging group, where the sol-gel derived material is the volume of acetone per mass of the dry sol-gel derived composition, when placed in excess acetone and the sol-gel derived material swellable to at least five times its dry mass, when placed in excess acetone, and where the amount of active material encapsulated by the porous sol-gel composition is from about 150 to about 1100% w/w.

### CROSS-REFERENCE TO RELATED APPLICATIONS

This application claims priority to U.S. Provisional Patent Application Ser. No. 62/159,165, entitled “ACTIVE MATERIALS ENCAPSULATED IN A SOL-GEL DERIVED COMPOSITION AND METHOD OF USE,” filed May 8, 2015, which is expressly incorporated by reference herein in its entirety.

### BACKGROUND

### 1. Field of the Invention

The present invention relates generally to the chemical arts. More particularly, the invention relates to active materials encapsulated in a sol-gel derived composition.

### 2. Discussion of Related Art

U.S. Pat. No. 7,790,830 and U.S. patent application Ser. No. 13/575,718, filed Aug. 2, 2102, disclose sol-gel compositions having a number of useful properties. For example, the sol-gel compositions swell up to about eight to ten times their original volume in the presence of a non-polar sorbate. There remains, however, a definite need for additional sol-gel compositions having improved properties including compositions that are substantially mesoporous, have increased swellability and/or produce a greater force upon swelling and, in particular, there remains a need for sol-gel derived composition that effectively encapsulate and release active materials.

### SUMMARY OF THE INVENTION

Now, in accordance with one aspect of the invention, there has been discovered an encapsulated active material product comprising: an active material encapsulated in a porous sol-gel derived material, the sol-gel derived material including a plurality of alkylsiloxy substituents and the sol-gel derived material obtained from:

(a) at least one first alkoxysilane precursor having the formula:

(R′O)₃—Si—(CH₂)ₙ—Ar—(CH₂)ₘ—Si—(OR′)₃ (1)

where n and m are individually an integer from 1 to 8, Ar is a single-, fused-, or poly-aromatic ring, and each R′ is independently a C₁ to C₅ alkyl group and

(b) optionally, at least one second precursor having the formula:

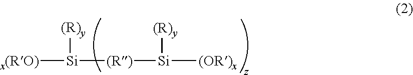

where x is 1, 2, 3 or 4; y is 0, 1, 2, 3; z is 0, 1; the total of x+y+z is 4; each R is independently an organic functional group; each an R′ is independently a C₁ to C₅ alkyl group and R″ is an organic bridging group, where the sol-gel derived material is swellable to at least five times its dry mass, when placed in excess acetone, and where the amount of active material encapsulated by the porous sol-gel composition is from about 150 to about 1100% w/w. In another, aspect of the invention, the plurality of alkylsiloxy groups have the formula:

—(O)_{w}—Si—(R₃)_{4-w} (3).

In one aspect of the invention, the sol-gel derived material is swellable to at least 10 times its dry mass, when placed in excess acetone. In another aspect of the invention, the sol-gel derived material has a pore volume of from about 0.9 mL/g to about 1.1 mL/g. In one aspect of the invention, the sol-gel derived material has a surface area from about 300 m²/g to about 600 m²/g and, in one embodiment, the sol-gel derived material has a surface area of from about 300 m²/g to about 600 m²/g

In one aspect of the invention, the amount of active material encapsulated by the porous sol-gel composition is from about 250 to about 950% w/w. And in another aspect of the invention, the amount of active material encapsulated by the porous sol-gel composition is from about 400 to about 700% w/w.

In another aspect of the invention, the encapsulated active material product is a fragrance-containing product. In one aspect, the encapsulated active material product is a perfume or a deodorant and, in one aspect, the product is a cosmetic, a cleanser or medicament.

In one aspect of the invention, the encapsulated active material product further comprises a carrier for the encapsulated active material. And in one aspect, the encapsulated active material product is ethanol, methanol, isopropanol, acetone, or hexane.

### DETAILED DESCRIPTION OF THE PREFERRED EMBODIMENTS

Particular embodiments of the invention are described below in considerable detail for the purpose of illustrating its principles and operation. However, various modifications may be made, and the scope of the invention is not limited to the exemplary embodiments described below.

Unless otherwise described, all technical terms used herein have the same meaning as commonly understood by one of ordinary skill in the art to which the present invention pertains.

As used herein, “mesopore” means a pore size between six and eighty nm.

As used herein, “micropore” means a pore size less than six nm.

As used herein “sorb” means to take up whether by adsorption, absorption, sequestration or a combination thereof.

As used herein, the term “solute” means any compound dissolved in a solvent.

As used herein, the term “sorbate” means an organic compound that is sorbed by the sol-gel derived composition by adsorption, absorption, or a combination thereof.

As used herein “swell” means the volume of solvent absorbed per mass of dry sol-gel derived composition.

As used herein, “nanoparticle” means a particle sized between about 0.05 and about 50 nanometers in one dimension.

In accordance with the invention, there has been discovered active materials encapsulated in a porous sol-gel derived composition. In one aspect of the invention, water-soluble organic liquids and water-soluble organic liquid solutions are encapsulated in a porous sol-gel composition.

In one aspect, the porous sol-gel composition is obtained from at least one first alkoxysilane precursor having the formula:

(RO)₃—Si—(CH₂)ₙ—Ar—(CH₂)ₘ—Si—(OR)₃ (1)

where n and m are individually an integer from 1 to 8, Ar is a single-, fused-, or poly-aromatic ring, such as a phenyl or naphthyl ring, and each R is independently a C₁ to C₅ alkyl, such as methyl or ethyl.

Exemplary first alkoxysilane precursors include, without limitation, bis(trialkoxysilylalkyl)benzenes, such as 1,4-bis(trimethoxysilylmethyl)benzene (BTB), bis(triethoxysilylethyl)benzene (BTEB), and mixtures thereof, with bis(triethoxysilylethyl)benzene being preferred.

In another aspect, the porous sol-gel composition is obtained from a mixture of the at least one first alkoxysilane precursor and at least one second alkoxysilane precursor, where the at least one second alkoxysilane precursor has the formula:

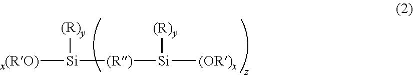

where x is 1, 2, 3 or 4; y is 0, 1, 2, 3; z is 0, 1; where the total of x+y+z is 4; R is independently an organic functional group; R′ is independently an alkyl group; and R″ is an organic bridging group, for example an alkyl or aromatic bridging group

In one aspect, x is 2 or 3, y is 1 or 2 and z is 0 and R′ is a methyl, an ethyl, or a propyl group. In another aspect, R comprises an unsubstituted or substituted straight-chain hydrocarbon group, branched-chain hydrocarbon group, cyclic hydrocarbon group, or aromatic hydrocarbon group.

In some embodiments, each R is independently an aliphatic or non-aliphatic hydrocarbon containing up to about 30 carbons, with or without one or more hetero atoms (e.g., sulfur, oxygen, nitrogen, phosphorous, and halogen atoms) or hetero atom-containing moieties. Representative R's include straight-chain hydrocarbons, branched-chain hydrocarbons, cyclic hydrocarbons, and aromatic hydrocarbons and are unsubstituted or substituted. In some aspects, R includes alkyl hydrocarbons, such as C₁-C₃ alkyls, and aromatic hydrocarbons, such as phenyl, and aromatic hydrocarbons substituted with heteroatom containing moieties, such —OH, —SH, —NH₂, and aromatic amines, such as pyridine.

Representative substituents for R include primary amines, such as aminopropyl, secondary amines, such as bis(triethoxysilylpropyl)amine, tertiary amines, thiols, such as mercaptopropyl, isocyanates, such as isocyanopropyl, carbamates, such as propylbenzylcarbamate, alcohols, alkenes, pyridine, halogens, halogenated hydrocarbons or combinations thereof.

Exemplary second alkoxysilane alkoxysilane precursors include, without limitation, tetramethoxysilane, methyltrimethoxysilane, methyltriethoxysilane, dimethyldimethoxysilane, dimethyldiethoxysilane, phenyltrimethoxysiliane, aminopropyltrimethoxysilane, (4-ethylbenzyl)trimethoxysilane, 1,6-bis(trimethoxysilyl)hexane, 1,4-bis(triethoxysilyl)benzene, bis(triethoxysilylpropyl)amine, 3-cyanopropyltrimethoxysilane, 3-sulfoxypropyltrimethoxysilane, isocyanopropyltrimethoxysilane, 2-(3,4-epoxycyclohexyl)ethyltrimethoxysilane, and

Examples of suitable second precursors include, without limitation, dimethyldimethoxysilane, (4-ethylbenzyl)trimethoxysilane, 1,6-bis(trimethoxysilyl)hexane, 1,4-bis(trimethoxysilyl)benzene, tetramethoxysilane, methyltrimethoxysilane, methyltriethoxysilane, phenyltrimethoxysilane, with dimethyldimethoxysilane, (4-ethylbenzyl)trimethoxysilane, and phenyltrimethoxysilane being preferred.

Other examples of useful second precursors include, without limitation, para-trifluoromethylterafluorophenyltrimethoxysilane, (tridecafluoro-1,1,2,2-tetrahydrooctyl)trimethoxysilane; second precursors having a ligand containing —OH, —SH, —NH2 or aromatic nitrogen groups, such as 2-(trimethoxysilylethyl)pyridine, 3-aminopropyltrimethoxysilane, 3-mercaptopropyltrimethoxysilane, and second precursors with protected amine groups, such as trimethoxypropylbenzylcarbamate.

In one aspect, the second alkoxysilane alkoxysilane precursor is dimethyldimethoxysilane, dimethyldiethoxysilane, phenyltrimethoxysilane or aminopropyltriethoxysilane.

It is a distinct advantage of the invention, that the properties of the sol-gel derived composition can be modified by the second precursor. The second alkoxysilane precursor can be selected to produce sol-gel compositions having improved properties. In one aspect, the sol-gel derived compositions are substantially mesoporous. In one aspect, the sol-gel derived compositions contain less than about 20% micropores and, in one aspect, the sol-gel derived compositions contain less than about 10% micropores. In one aspect, the mesopores have a pore volume greater than 0.50 mL/g as measured by the BET/BJH method and in one aspect, the mesopores have a pore volume greater than 0.75 mL/gas measured by the BET/BJH method. In another aspect, the sol-gel derived composition generates a force upon swelling that is greater than about 200 N/g as measured by swelling with acetone in a confined system; in one aspect, the sol-gel derived composition generates a force upon swelling that is greater than about 400 N/g as measured by swelling with acetone in a confined system and in one aspect one aspect, the sol-gel derived composition generates a force upon swelling that is greater than about 700 N/g as measured by swelling with acetone in a confined system.

And in another aspect, the sol-gel derived compositions absorb at least ten times the volume of acetone per mass of dry sol-gel derived composition.second. Examples of second precursors useful to effect the swellability of the sol-gel derived composition include dimethyldimethoxysilane, (4-ethylbenzyl)trimethoxysilane, 1,6-bis(trimethoxysilyl)hexane, 1,4-bis(trimethoxysilyl)benzene methyltrimethoxysilane, phenyltrimethoxysilane, with dimethyldimethoxysilane, (4-ethylbenzyl)trimethoxysilane, and phenyltrimethoxysilane being preferred.

The porous sol-gel compositions are obtained from an alkoxysilane precursor reaction medium, under acid or base sol-gel conditions, preferably base sol-gel conditions. In one aspect of the present invention, the alkoxysilane precursor reaction medium contains from about 100:00 vol:vol to about 10:90 vol:vol of the at least one first alkoxysilane precursor to the at least one second alkoxysilane precursor, in one aspect, and from about 20:80 vol:vol to about 50:50 vol:vol first alkoxysilane precursor to second alkoxysilane precursor. In one aspect, the alkoxysilane precursor reaction medium contains 100% of the at least one first alkoxysilane alkoxysilane precursor. The relative amounts of the at least one first alkoxysilane and the at least one second alkoxysilane alkoxysilane precursors in the reaction medium will depend on the particular alkoxysilane precursors and the particular application for the resulting sol-gel composition.

The reaction medium includes a solvent for the alkoxysilane precursors. In some aspects, the solvent has a Dimoth-Reichart solvatochromism parameter (E_{T}) between 170-205 kJ/mol. Suitable solvents include, without limitation, tetrahydrofuran (THF), acetone, dichloromethane/THF mixtures containing at least 15% by vol. THF, and THF/acetonitrile mixtures containing at least 50% by vol. THF. Of these exemplary solvents, THF is preferred. The alkoxysilane precursors are preferably present in the reaction medium at between about 0.25M and about 1M, more preferably between about 0.4M and about 0.8M, most preferably about 0.5 M.

A catalytic solution comprising a catalyst and water is rapidly added to the reaction medium to catalyze the hydrolysis and condensation of the alkoxysilane precursors, so that a sol gel coating is formed on the particles. Conditions for sol-gel reactions are well-known in the art and include the use of acid or base catalysts. Preferred conditions are those that use a base catalyst. Exemplary base catalysts include, without limitation, tetrabutyl ammonium fluoride (TBAF), fluoride salts, including but not limited to potassium fluoride, 1,5-diazabicyclo[4.3.0]non-5-ene (DBN), and alkylamines, including but not limited to propyl amines, of which TBAF is preferred.

As noted above, acid catalysts can be used to form sol-gel coatings, although acid catalysts are less preferred. Exemplary acid catalysts include, without limitation, any strong acid such as hydrochloric acid, phosphoric acid, sulfuric acid and the like.

In one aspect, water is present in the reaction medium at an amount so there is at least one half mole of water per mole of alkoxysilane groups in the alkoxysilane precursors. In one aspect, temperatures at polymerization can range from between the freezing point of the reaction medium up to the boiling point of the reaction medium. And in one aspect, the temperature range is from about 4° C. to about 50° C.

After gellation, the sol-gel coating is preferably aged for an amount of time suitable to induce syneresis, which is the shrinkage of the gel that accompanies solvent evaporation. The aging drives off much, but not necessarily all, of the solvent. While aging times vary depending upon the catalyst and solvent used to form the gel, aging is typically carried out for about 15 minutes up to about 10 days. In one aspect, aging is carried out for at least about 1 hour and, in one aspect, aging is carried out for about 2 to about 10 days. In one aspect, aging temperatures can range from between the freezing point of the solvent or solvent mixture up to the boiling point of the solvent or solvent mixture. And in one aspect, the aging temperature is from about 4° C. to about 50° C. And in some aspects, aging is carried out either in open atmosphere, under reduced pressure, in a container or oven.

After gellation and aging have been completed, the sol-gel composition is rinsed using an acidic solution, with solutions comprising stronger acids being more effective. In one aspect, the rinsing agent comprises concentrations between 0.009-0.2% w/v acid in an organic solvent. Representative organic solvents include solvents for the alkoxysilane precursors, including solvents having a Dimoth-Reichart solvatochromism parameter (ET) between 170-205 kJ/mol. Suitable solvents for use with the base catalysts include, without limitation, tetrahydrofuran (THF), acetone, dichloromethane/THF mixtures containing at least 15% by vol. THF, and THF/acetonitrile mixtures containing at least 50% by vol. THF. Preferred rinse reagents, include with out limitation, 0.01% wt:vol HCl or 0.01% wt:vol H2SO4 in acetone. In one aspect, the sol-gel composition is rinsed with the acidic solution for at least 5 min. And in one aspect, the sol-gel composition is rinsed for a period of time of from about 0.5 hr to about 12 hr.

An alternative rinsing method is to use a pseudo-solvent system, such as supercritical carbon dioxide.

After rinsing, the sol-gel derived material is characterized by the presence of residual silanols. In one aspect, the silanol groups are derivatized with a reagent in an amount sufficient to stoichiometrially react with the residual silanols and prevent cross-linking that might otherwise occur between the residual silanol groups. Suitable derivatization reagents include, without limitation, reagents that have both one or more silanol-reactive groups and one or more non-reactive alkyl groups. The derivatization process results in the end-capping of the silanol-terminated polymers present within the sol-gel derived material with alkylsiloxy groups having the formula:

—(O)_{w}—Si—(R₃)_{4-w} (3)

where each R₃ is independently an organic functional group as described above and w is an integer from 1 to 3.

One suitable class of derivatization reagents includes halosilanes, such as monohalosilane, dihalosilane and trihalosilane derivatization reagents that contain at least one halogen group and at least one alkyl group R₃, as described above. The halogen group can be any halogen, preferably Cl, Fl, I, or Br. Representative halosilanederivatization reagents include, without limitation, chlorosilanes, dichlorosilanes, fluorosilanes, difluorosilanes, bromosilanes, dibromosilanes, iodosilanes, and di-iodosilanes. Exemplary halosilanes suitable for use as derivatization reagents include, without limitation, cynanopropyldimethylchlorosilane, phenyldimethylchlorosilane, chloromethyldimethylchlorosilane, (trideca-fluoro-1,1,2,2-tertahydro-octyl)dimethylchlorosilane, n-octyldimethylchlorosilane, and n-octadecyldimethylchlorosilane. And in one aspect, the halosilane derivatization reagent is trimethyl chlorosilane.

Another suitable class of derivatization reagents includes silazanes or disilazanes. Any silazane with at least one reactive group and at least one alkyl group R₃, as described above can be used. A preferred disilazane is hexamethyldisilazane.

The sol-gel derived composition is preferably rinsed in any of the rinsing agents described above to remove excess derivatization reagent, and then dried. Drying can be carried out under any suitable conditions, but preferably in an oven, e.g., for about 2 hours at about 60 C to produce the porous, swellable, sol-gel derived composition.

In some aspects, the compositions contain a plurality of flexibly tethered and interconnected organosiloxane particles having diameters on the nanometer scale. The organosiloxane nanoparticles form a porous matrix defined by a plurality of aromatically cross-linked organosiloxanes that create a porous structure.

And in some aspects, the resulting sol-gel compositions are hydrophobic, resistant to absorbing water, and absorb at least ten times the volume of acetone per mass of dry sol-gel derived composition. Without being bound by theory, it is believed that swelling is derived from the morphology of interconnected organosilica particles that are cross-linked during the gel state to yield a porous material or polymeric matrix. Upon drying the gel, tensile forces are generated by capillary-induced collapse of the polymeric matrix. This stored energy can be released as the matrix relaxes to an expanded state when a sorbate disrupts the inter-particle interactions holding the dried material in the collapsed state.

In one aspect, the resulting sol-gel composition contains a plurality of flexibly tethered and interconnected organosiloxane particles having diameters on the nanometer scale. The organosiloxane nanoparticles form a porous matrix defined by a plurality of aromatically cross-linked organosiloxanes that create a porous structure. In some aspects, the resulting sol-gel composition has a pore volume of from about 0.9 mL/g to about 1.1 mL/g and, in some aspects, a pore volume of from about 0.2 mL/g to about 0.6 mL/g. In some aspects, the resulting sol-gel composition has a surface area of from about 300 m²/g to about 600 m²/g and, in some aspects, a surface area of from about 600 m²/g to about 1000 m²/g.

And one aspect, the resulting sol-gel composition is hydrophobic, resistant to absorbing water, and swellable to at least two times its mass, when dry, in acetone. In one aspect, the sol-gel composition is swellable to at least five times its dry mass, when placed in excess acetone and, in one aspect, the sol-gel composition is swellable to at least ten times its dry mass, when placed in excess acetone. Useful sol-gel compositions include, but are not limited to, OSORB® media available from ABSMaterials, Wooster, Ohio.

It is a distinct advantage that the porous sol-gel composition can be used to encapsulate a large number of active materials. In one aspect, the active material is a biologically active species. In one embodiment, the active ingredient is an essential oil, pharmaceutical, vitamin, herbicide pesticide, flavorant or the like In another aspect, the active material is a volatile organic liquid, such as ethanol a fragrance or the like.

In one aspect, the active material is a gas, liquid or solid mixed with an organic liquid carrier to form a solution. Representative organic liquid carriers include, without limitation, ethanol, methanol, isopropanol, acetone, and hexane.

In one aspect, the active material is present in the solution at a concentration of at least 25 grams per liter; in one aspect, the active material is present in the solution at a concentration of at least 50 grams per liter; and in one aspect, the active material is present in the solution at a concentration of at least 100 grams per liter.

The active materials can be encapsulated by any suitable method. In one aspect, the active materials are encapsulated by contacting the porous sol-gel composition with the active materials under conditions sufficient to cause the porous sol-gel composition to sorb the active materials. It is a definite advantage of the inventive method that the active materials can be sorbed by the porous sol-gel composition at ambient temperature and pressure.

In one aspect, the amount of active materials encapsulated by the porous sol-gel composition is from about 150 to about 1100% w/w. In a further aspect, the amount of active materials encapsulated is from about 250 to about 950% w/w. And is a still further aspect, the amount of active materials encapsulated is from about 400 to about 700% w/w. Representative encapsulated active material products, include, but are not limited to perfumes, cosmetics, personal care products (e.g., deodorants), cleansers (e.g., skin cleansers), and medicaments, including pharmaceuticals in the form of tablets, capsules, ointments, or the like.

The compositions in accordance with the invention can be used alone or formulated with other ingredients. Representative formulations include, but are not limited fragrances, cosmetics, cleansers (e.g., skin cleansers), and medicaments, including pharmaceuticals in the form of tablets, capsules, ointments, or the like.

Encapsulated active ingredients according to the invention can be contained in a wide variety of fragrance-containing compositions. Representative compositions include, without limitation, in any suitable personal care product, such as perfumes, skincare products, including without limitation, body washes, face washes, body oils, body lotions or creams, anti-aging creams or lotions, body gels, day creams or lotions, night creams or lotions, treatment creams, skin protection ointments, moisturizing gels, body milks, suntan lotions, suntan creams, self-tanning creams, artificial tanning compositions, cellulite gels, peeling preparations, facial masks, depilatories, shaving creams, deodorants, anti-perspirants, and the like, particularly for topical administration.

Representative fragrances include anethol, methyl heptine carbonate, ethyl aceto acetate, para cymene, nerol, decyl aldehyde, para cresol, methyl phenyl carbinyl acetate, ionone alpha, ionone beta, undecylenic aldehyde, undecyl aldehyde, 2,6-nonadienal, nonyl aldehyde, octyl aldehyde, phenyl acetaldehyde, anisic aldehyde, benzyl acetone, ethyl-2-methyl butyrate, damascenone, damascone alpha, damascone beta, flor acetate, frutene, fructone, herbavert, iso cyclo citral, methyl isobutenyl tetrahydro pyran, isopropyl quinoline, 2,6-nonadien-1-ol, 2-methoxy-3-(2-methylpropyl)-pyrazine, methyl octine carbonate, tridecene-2-nitrile, allyl amyl glycolate, cyclogalbanate, cyclal C, melonal, gamma nonalactone, c is 1,3-oxathiane-2-methyl-4-propyl, benzaldehyde, benzyl acetate, camphor, carvone, borneol, bornyl acetate, decyl alcohol, eucalyptol, linalool, hexyl acetate, iso-amyl acetate, thymol, carvacrol, limonene, menthol, iso-amyl alcohol, phenyl ethyl alcohol, alpha pinene, alpha terpineol, citronellol, alpha thujone, benzyl alcohol, beta gamma hexenol, dimethyl benzyl carbinol, phenyl ethyl dimethyl carbinol, adoxal, allyl cyclohexane propionate, beta pinene, citral, citronellyl acetate, citronellal nitrile, dihydro myrcenol, geraniol, geranyl acetate, geranyl nitrile, hydroquinone dimethyl ether, hydroxycitronellal, linalyl acetate, phenyl acetaldehyde dimethyl acetal, phenyl propyl alcohol, prenyl acetate, triplal, tetrahydrolinalool, verdox, and cis-3-hexenyl acetate.

Other representative fragrances include ethyl phenyl glycidate, ethyl vanillin, heliotropin, indol, methyl anthranilate, vanillin, amyl salicylate, coumarin, ambrox, bacdanol, benzyl salicylate, butyl anthranilate, cetalox, ebanol, cis-3-hexenyl salicylate, lilial, gamma undecalactone, gamma dodecalactone, gamma decalactone, calone, cymal, dihydro iso jasmonate, iso eugenol, lyral, methyl beta naphthyl ketone, beta naphthol methyl ether, para hydroxyl phenyl butanone, 8-cyclohexadecen-1-one, oxocyclohexadecen-2-one/habanolide, florhydral, intreleven aldehyde eugenol, amyl cinnamic aldehyde, hexyl cinnamic aldehyde, hexyl salicylate, methyl dihydro jasmonate, sandalore, veloutone, undecavertol, exaltolide/cyclopentadecanolide, zingerone, methyl cedrylone, sandela, dimethyl benzyl carbinyl butyrate, dimethyl benzyl carbinyl isobutyrate, triethyl citrate, cashmeran, phenoxy ethyl isobutyrate, iso eugenol acetate, helional, iso E super, ionone gamma methyl, pentalide, galaxolide, phenoxy ethyl propionate.

The fragrances can comprise a pre-formed blend, either extracted from natural products, or possibly created synthetically. Representatives of such pre-formed blends include oils from:—Bergamot, cedar atlas, cedar wood, clove, geranium, guaiac wood, jasmine, lavender, lemongrass, lily of the valley, lime, neroli, musk, orange blossom, patchouli, peach blossom, petitgrain or petotgrain, pimento, rose, rosemary, and thyme.

In one aspect of the invention, the composition preferably contains an antiperspirant active. Antiperspirant actives are preferably incorporated in an amount of from 0.5-50%, particularly from 5 to 30% and especially from 10% to 26% of the weight of the composition. It is often considered that the main benefit from incorporating of up to 5% of an antiperspirant active in a stick composition is manifest in reducing body odour, and that as the proportion of antiperspirant active increases, so the efficacy of that composition at controlling perspiration increases.

Antiperspirant actives for use herein are often selected from astringent active salts, including in particular aluminium, zirconium and mixed aluminium/zirconium salts, including both inorganic salts, salts with organic anions and complexes. Preferred astringent salts include aluminium, zirconium and aluminium/zirconium halides and halohydrate salts, such as chlorohydrates.

Aluminium halohydrates are usually defined by the general formula Al₂(OH)ₓQ_{y}wH2 in which Q represents chlorine, bromine or iodine, x is variable from 2 to 5 and x+y=6 while wH₂O represents a variable amount of hydration. Especially effective aluminium halohydrate salts, known as activated aluminium chlorohydrates, are described in EP-A-6739 (Unilever N V et al).

Zirconium actives can usually be represented by the empirical general formula: ZrO(OH)₂n-nzB_{z}.wH₂O in which z is a variable in the range of from 0.9 to 2.0 so that the value 2n-nz is zero or positive, n is the valency of B, and B is selected from the group consisting of chloride, other halide, sulphamate, sulphate and mixtures thereof. Possible hydration to a variable extent is represented by wH₂O. Preferable is that B represents chloride and the variable z lies in the range from 1.5 to 1.87. In practice, such zirconium salts are usually not employed by themselves, but as a component of a combined aluminium and zirconium-based antiperspirant.

Antiperspirant complexes based on the above-mentioned astringent aluminium and/or zirconium salts can be employed. The complex often employs a compound with a carboxylate group, and advantageously this is an amino acid. Examples of suitable amino acids include dl-tryptophan, dl-.beta.-phenylalanine, dl-valine, dl-methionine and .beta.-alanine, and preferably glycine.

In one aspect complexes of a combination of aluminium halohydrates and zirconium chlorohydrates together with amino acids such as glycine, which are disclosed in U.S. Pat. No. 3,792,068 (Luedders et al).

The proportion of solid antiperspirant salt in a suspension (anhydrous) composition normally includes the weight of any water of hydration and any complexing agent that may also be present in the solid active.

For incorporation of compositions according to the present invention, desirably at least 90%, preferably at least 95% and especially at least 99% by weight of the particles have a diameter in the range of from 0.1 μm p to 100 μm, and usually have an average particle diameter of at least 1 μm and especially below 20 μm. In some highly desirable contact compositions the particles by weight have a weight average particle size of at least 2 μm and particularly below 10 μm, such as in the range of from 3 to 8 μm.

Compositions according to the invention may be emulsions. In such compositions, the antiperspirant active is commonly dissolved in the aqueous phase, commonly at a weight concentration in that phase of between 10 and 55%. In many suitable emulsions, the concentration of antiperspirant active is chosen in relation to the weight of oils (including any non-encapsulated fragrance oils), decreasing progressively from a ratio of about 3:1 to 5:1 when the proportion of oils is below 10% to a ratio in the range of 3:2 to 2:3 when the oils content is at least 50% of the total weight of the composition (excluding any propellant).

The invention compositions may include one or more thickeners or gellants (sometimes called structuring or solidifying agents) to increase the viscosity of or solidify the liquid carrier in which the particulate materials are suspended as is appropriate for application from respectively soft solid (anhydrous cream) dispensers or stick dispensers.

Compositions according to the invention may be stick compositions. Such compositions desirably have a hardness as measured in a conventional penetration test (Seta) of less than 30 mm, preferably less than 20 mm and particularly desirably less than 15 mm. Many have a penetration of from 7 to 13 or 7.5 to 10 or 12.5 mm. The conventional penetration test employed herein, utilises a lab plant penetrometer equipped with a Seta wax needle (weight 2.5 grams) which has a cone angle at the point of the needle specified to be 9 degree. 10′+/−15′. A sample of the composition with a flat upper surface is used. The needle is lowered onto the surface of the composition and then a penetration hardness measurement is conducted by allowing the needle with its holder to drop under the combined weight of needle and holder of 50 grams for a period of five seconds after which the depth of penetration is noted. Desirably the test is carried out at six points on each sample and the results are averaged.

The gellants for forming stick compositions herein are usually selected from one or more of two classes: non-polymeric fibre-forming gellants and waxes, optionally supplemented by incorporation of a particulate silica and/or an oil-soluble polymeric thickener.

Waxes, when employed, are often selected from hydrocarbons, linear fatty alcohols, silicone polymers, esters of fatty acids or mixtures containing such compounds along with a minority (less than 50% w/w and often less than 20% w/w) of other compounds.

Non-polymeric fibre-forming gellants, when employed, are typically dissolved in a water-immiscible blend of oils at elevated temperature and on cooling precipitate out to form a network of very thin strands that are typically no more than a few molecules wide. One particularly effective category of such thickeners comprises N-acyl aminoacid amides and in particular linear and branched N-acyl glutamic acid dialkylamides, such as in particular N-lauroyl glutamic acid di n-butylamide and N-ethylhexanoyl glutamic acid di n-butylamide and especially mixtures thereof. Such amido gellants can be employed in anhydrous compositions according to the present invention, if desired, with 12-hydroxystearic acid.

A gellant is often employed in a stick or soft solid composition at a concentration of from 1.5 to 30%, depending on the nature of the gellant or gellants, the constitution of the oil blend and the extent of hardness desired.

The anhydrous compositions can contain one or more optional ingredients, such as one or more of those selected from those identified below.

Optional ingredients include wash-off agents, often present in an amount of up to 10% w/w to assist in the removal of the formulation from skin or clothing. Such wash-off agents are typically nonionic surfactants such as esters or ethers containing a C.sub.8 to C.sub.22 alkyl moiety and a hydrophilic moiety which can comprise a polyoxyalkylene group (POE or POP) and/or a polyol.

The compositions herein can incorporate one or more cosmetic adjuncts. Such adjuncts can include skin feel improvers, such as talc or finely divided (i.e. high molecular weight) polyethylene, i.e. not a wax, for example Accumist™, in an amount of 1 up to about 10%; a moisturiser, such as glycerol or polyethylene glycol (mol wt 200 to 600), for example in an amount of up to about 5%; skin benefit agents such as allantoin or lipids, for example in an amount of up to 5%; colours; skin cooling agents other than the already mentioned alcohols, such a menthol and menthol derivatives, often in an amount of up to 2%, all of these percentages being by weight of the composition. A further optional ingredient comprises a preservative, such as ethyl or methyl parabens or BHT (butyl hydroxy toluene) such as in an amount of from 0.01 to 0.1% w/w.

In another aspects encapsulated active ingredients according to the invention can be contained in a cosmetic preparations. Representative formulations include, but are not limited to, skin-care preparations, e.g. skin emulsions, multi-emulsions or skin oils and body powders; cosmetic personal care preparations, e.g. facial make-up in the form of lipsticks, lip gloss, eye shadow, liquid make-up, day creams or powders, facial lotions, creams and powders (loose or pressed); and light-protective preparations, such as sun tan lotions, creams and oils, sun blocks and pro-tanning preparations.

In one aspect, the compositions according to the invention comprise a liquid or non liquid cosmetically acceptable carrier to act as a diluent, dispersant or vehicle for the sorbate-loaded sol-gel derived composition, so as to facilitate its distribution when the composition is applied to the skin. Carriers other than or in addition to water can include liquid or solid emollients, solvents, humectants, thickeners and powders. Particularly suitable non aqueous carriers include polydimethyl siloxane and/or polydimethyl phenyl siloxane. Such formulations can additionally include colorants, sequestering agents, thickening or solidifying (consistency legislating) agents, emollients, UV absorbers, skin-protective agents, antioxidants and preservatives.

And in one aspect, the encapsulated active materials are formulated in at least one liquid or non liquid pharmaceutically acceptable carrier. As used herein, “pharmaceutically acceptable carrier” encompasses any of the standard pharmaceutical carriers known to those of skill in the art. Pharmaceutically acceptable carriers include solvent(s), vehicle(s), adjuvant(s), excipient(s), binder(s), thickener(s), suspending agent(s), or filler substance(s) that are known to tire skilled artisan suitable for administration to human and/or animals. Other useful carriers include gum acacia, agar, petrolatum, lanolin, dimethyl sulfoxide (DMSO), normal saline (NS), phosphate buffered saline (PBS), sodium alginate, bentonite, carbomer, carboxymethylcellulose, carrageenan, powdered cellulose, cholesterol, gelatin, hydroxyethyl cellulose, hydroxypropyl cellulose, hydroxypropyl methylcellulose, methylcellulose, octoxynol 9, oleyl alcohol, polyvinyl alcohol, povidone, propylene glycol monostearate, sodium lauryl sulfate, sorbitan esters, stearyl alcohol, tragacanth, xanthan gum, chondrus, glycerin, trolamine, avocado oil, almond oil, coconut oil, coconut butter, propylene glycol, ethyl alcohol, malt, and malt extract.

Medicants include medicaments taken into the bodies of humans or non-human, vertebrate animals, or applied topically thereto, by a delivery system. A medicament is a therapeutic agent or substance, such as a drug, medicine, irrigant, bandage, or other medical or dental device, that promotes recovery from injury or ailment or prevents or alleviates the symptoms of disease. Medicaments containing the sorbate-loadel sol-gel derived composition can be formulated for any suitable systemic or non-systemic delivery system, including delivery systems for oral, enteral, or parenteral delivery routes include tablets, troches, lozenges, aqueous or oily suspensions, dispersible powders or granules, emulsions, hard or soft capsules, syrups, beverages, elixirs or enteral formulas, lavage or enema solutions, adhesive patches, infusions, injectates, intravenous drips, inhalants, or implants. Delivery systems also include topical creams, gels, suppositories, or ointments for non-systemic localized delivery or systemic delivery via the blood stream.

Systemic delivery systems that are contemplated by the present invention include, but are not limited to, implant; adhesive transdermal patches; topical creams, gels or ointments for transdermal delivery, transmucosal delivery matrices or suppositories or gels. It is contemplated that tire compositions of the present invention are formulated to deliver an effective amount of the sorbate by these or any other pharmaceutically acceptable systemic delivery system.

In accordance the inventive method, the active materials are subsequently released from the porous sol-gel derived composition. In one aspect, the active material, such as a fragrance is released by natural diffusion. In another aspect, the active materials are released by applying heat to the encapsulated material, such as the enhanced diffusion of an herbicide via thermal desorption And in another aspect, the active materials are displaced by contacting the encapsulated material with another substance, such as displacement and release of aromatherapeutic fragrances from the sol-gel derived composition upon absorption of malodors by the sol-gel derived composition or the timed release of a pharmaceutical by diffusion into water.

Distinctive advantages of the encapsulated material include (1) the stability of the sol-gel derived composition, (2) the high loading capacity of the sol-gel derived composition; (3) the prevention of biological degradation of the active materials, (4) the protection of active materials that are water- or UV-sensitive, and (5) the slow and/or controlled release of the active ingredients due to the sol-gel derived material.

We claim:

1. An encapsulated active material product comprising:
an active material encapsulated in a porous sol-gel derived material, the sol-gel derived material including a plurality of alkylsiloxy substituents and the sol-gel derived material obtained from:
(a) at least one first alkoxysilane precursor having the formula:
(R′O)₃—Si—(CH₂)ₙ—Ar—(CH₂)ₘ—Si—(OR′)₃ (1)
where n and m are individually an integer from 1 to 8, Ar is a single-, fused-, or poly-aromatic ring, and each R′ is independently a C₁ to C₅ alkyl group and
(b) optionally, at least one second precursor having the formula: 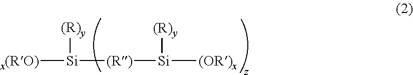
where x is 1, 2, 3 or 4; y is 0, 1, 2, 3; z is 0, 1; the total of x+y+z is 4; each R is independently an organic functional group; each an R′ is independently a C₁ to C₅ alkyl group and R″ is an organic bridging group, where the sol-gel derived material is swellable to at least five times its dry mass, when placed in excess acetone, and where the amount of active material encapsulated by the porous sol-gel composition is from about 150 to about 1100% w/w.

2. The encapsulated active material product of claim 1 wherein the plurality of alkylsiloxy groups have the formula:
—(O)_{w}—Si—(R₃)_{4-w} (3)
where each R₃ is independently an organic functional group and w is an integer from 1 to 3

3. The encapsulated active material product of claim 1 wherein the sol-gel derived material is swellable to at least ten times its dry mass, when placed in excess acetone.

4. The encapsulated active material product of claim 1 wherein the sol-gel derived material has a pore volume of from about 0.9 mL/g to about 1.1 mL/g.

5. The encapsulated active material product of claim 1 wherein the sol-gel derived material has a surface area of from 300 m²/g to about 600 m²/g

6. The encapsulated active material product of claim 1 wherein the sol-gel derived material has a surface area of from 300 m²/g to about 600 m²/g

7. The encapsulated active material product of claim 1 wherein the amount of active material encapsulated by the porous sol-gel composition is from about 250 to about 950% w/w.

8. The encapsulated active material product of claim 1 wherein the amount of active material encapsulated by the porous sol-gel composition is from about 400 to about 700% w/w.

9. The encapsulated active material product of claim 1 wherein the product is a fragrance-containing product.

10. The encapsulated active material product of claim 1 wherein the product is a perfume or a deodorant.

11. The encapsulated active material product of claim 1 wherein the product is a cosmetic, a cleanser or medicament.

12. The encapsulated active material product of claim 1 wherein the product further comprises a carrier for the encapsulated active material.

13. The encapsulated active material product of claim 12 wherein the carrier is ethanol, methanol, isopropanol, acetone, and hexane.

14. An encapsulated active material product comprising:
an active material encapsulated in a porous sol-gel derived material, the sol-gel derived material including a plurality of alkylsiloxy substituents and the sol-gel derived material obtained from:
(a) at least one first alkoxysilane precursor having the formula:
(R′O)₃—Si—(CH₂)ₙ—Ar—(CH₂)ₘ—Si—(OR′)₃ (1)
where n and m are individually an integer from 1 to 8, Ar is a single-, fused-, or poly-aromatic ring, and each R′ is independently a C₁ to C₅ alkyl group and
(b) optionally, at least one second precursor having the formula: 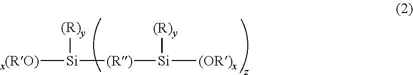
where x is 1, 2, 3 or 4; y is 0, 1, 2, 3; z is 0, 1; the total of x+y+z is 4; each R is independently an organic functional group; each an R′ is independently a C₁ to C₅ alkyl group and R″ is an organic bridging group, where the plurality of alkylsiloxy groups have the formula:
—(O)_{w}—Si—(R₃)_{4-w} (3)
where each R₃ is independently an organic functional group and w is an integer from 1 to 3, where the sol-gel derived material is swellable to at least 10 times its dry mass, when placed in excess acetone, where the sol-gel derived material has a pore volume of from about 0.9 mL/g to about 1.1 mL/g and where the amount of active material encapsulated by the porous sol-gel composition is from about 250 to about 950% w/w. 3.

15. The encapsulated active material product of claim 14 wherein the sol-gel derived material has a pore volume of from about 0.2 mL/g to about 0.6 mL/g.

16. The encapsulated active material product of claim 14 wherein the amount of active material encapsulated by the porous sol-gel composition is from about 400 to about 700% w/w.

17. The encapsulated active material product of claim 14 wherein the product a fragrance-containing product.

18. The encapsulated active material product of claim 141 wherein the product a perfume or a deodorant.

19. An encapsulated fragrance-containing product comprising:
fragrance-containing encapsulated in a porous sol-gel derived material, the sol-gel derived material including a plurality of alkylsiloxy substituents and the sol-gel derived material obtained from:
(a) at least one first alkoxysilane precursor having the formula:
(R′O)₃—Si—(CH₂)ₙ—Ar—(CH₂)ₘ—Si—(OR′)₃ (1)
where n and m are individually an integer from 1 to 8, Ar is a single-, fused-, or poly-aromatic ring, and each R′ is independently a C₁ to C₅ alkyl group and
(b) optionally, at least one second precursor having the formula: 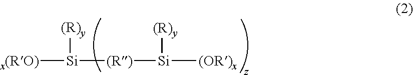
where x is 1, 2, 3 or 4; y is 0, 1, 2, 3; z is 0, 1; the total of x+y+z is 4; each R is independently an organic functional group; each an R′ is independently a C₁ to C₅ alkyl group and R″ is an organic bridging group, where the plurality of alkylsiloxy groups have the formula:
—(O)_{w}—Si—(R₃)_{4-w} (3)
where each R₃ is independently an organic functional group and w is an integer from 1 to 3, where the sol-gel derived material is the swellable to at least 10 times its dry mass, when placed in excess acetone, where the sol-gel derived material has a pore volume of from about 0.9 mL/g to about 1.1 mL/g and where the amount of fragrance-containing material encapsulated by the porous sol-gel composition is from about 250 to about 950% w/w.

20. The encapsulated active material product of claim 1 wherein the product a perfume or a deodorant.
